(19) United States
(12) Patent Application Publication
(10) Pub. No.: US 2022/0112535 A1
(43) Pub. Date: Apr. 14, 2022

(54) PRODUCTION OF PROTEIN WITH HUMANIZED N-GLYCOSYLATION IN INSECT CELLS

(71) Applicant: EXPRES2ION BIOTECHNOLOGIES APS, Hørsholm (DK)

(72) Inventors: Stine Broch Clemmensen, Hørsholm (DK); Magdalena Skrzypczak, Vanløse (DK); Teit Max Moscote Søgaard, Kokkedal (DK); Willem Adriaan De Jongh, Holte (DK)

(21) Appl. No.: 17/421,902

(22) PCT Filed: Jan. 10, 2020
(86) PCT No.: PCT/EP2020/050592

§ 371 Date: Jul. 9, 2021

(30) Foreign Application Priority Data
Jan. 10, 2019 (EP) 19151233.4

(51) Int. Cl. C12P 21/00; C12N 5/07

(52) U.S. Cl. CPC C12P 21/005; C12N 5/0601

(57) ABSTRACT

The present disclosure provides genetically modified insect cells that can produce glycosylated expression products having a human-like glycosylation pattern. In particular, the cells comprise disruption of the fdl and/or FucT6 genes. Also provided is expression systems and methods for recombinant protein production.

### FIELD OF THE INVENTION

The present invention relates to the field of recombinant production of protein. In particular, the present invention pertains to recombinant production in insect cells, such as Drosophila cells, of N-glycosylated antigens, which carry a humanized N-glycosylation pattern. The invention thus relates to methods of recombinant protein production as well as to expression vectors and adapted cell lines for this purpose.

### BACKGROUND OF THE INVENTION

Protein therapeutics such as monoclonal antibodies (mAbs), peptides and recombinant proteins, represent a large group of developing products in the biopharmaceutical industry. The majority of biological EMA and FDA-approved products are recombinant glycoproteins, which are used in treatment against a range of diseases, such as metabolic disorders, autoimmune diseases, and cancer. These products are produced in a wide variety of platforms, such as mammalian expression systems, including CHO and human cell lines, and non-mammalian expression systems, such as bacterial, yeast, plant and insect.

The most appropriate expression system for a specific therapeutic protein depends on the particular protein to be expressed and the intended usage. In the past, proteins with therapeutic capabilities were derived directly from the source, for example humans or pigs. Examples hereof are insulin, which was derived from pancreatic tissue and albumin derived from blood plasma. However, assuring reproducibility, purity, and safety was difficult with the emergence of genetic engineering technology this led to the development of recombinant expression systems for protein production.

Different expression systems have different advantages, but certain therapeutics have glycosylation requirements that currently mean that primarily CHO and other mammalian systems can be used for their production. Other expression systems have advantages like speed and ability to produce difficult proteins.

Glycosylation is generally important to consider when producing vaccine antigens. Especially N-linked glycans are important, as they influence glycoprotein half-life, dictate migration, ensure protein stability, and mediate cell signalling.

The most common expression systems and their glycosylation characteristics are described in the following.

Bacteria as an Expression System

In 1982 the first recombinant biopharmaceutical was approved. This was insulin (by Eli Lilly & Co.'s Humulin®) and it was produced in Escherichia coli. E. coli has since been used to produce commercially approved non-glycosylated therapeutic proteins, such as cytokines, and monoclonal antibodies and enzymes. Bacteria generally do not glycosylate proteins, as they lack glycosylation machinery. Due to the inability to add N-glycans to proteins, bacteria have a limitation in production compared with more complex hosts for proteins that require post-translational modifications (PTMs). However, the bacterium Campylobacter jejuni has exhibited a glycosylation machinery, which has successfully been transferred to E. coli. Although this is highly relevant for recombinant protein production, additional optimizations to establish a cost-effective process are still needed.

Yeast as an Expression System

Yeast-based systems have been extensively used for recombinant protein expression. Yeast and filamentous fungi offer numerous advantages as recombinant protein expression systems when compared with mammalian cell culture, including high recombinant protein titers, short fermentation times, and the ability to grow in chemically defined media. Saccharomyces cerevisiae is the expression system for close to 20% of all biopharmaceuticals, including insulin, hepatitis vaccines, and human serum albumin. Yeasts can be grown in industrial scale and they represent very robust expression, capable of folding proteins and secreting these into the medium. Furthermore, they also present well characterized N-glycosylation, which is often hyper-mannosylation. To produce better drugs many efforts have been made to humanize the N-glycosylation in yeasts, and in 2006 Hamilton et al managed to construct a Pichia pastoris cell line that adds 90.5% of double-sialylated N-glycan structures on purified erythropoietin (EPO).

Plant Cells as an Expression System

Plant cells can be cultured in basal culture medium and are easily scaled up. Plant cells do not contain endotoxins like E. coli and they do not represent the same disadvantages as recombinant protein production in whole plants. Plant cells show greater similarity to human N-glycans than yeast does. However, plant cells are also known to express α1,3-fucose and β1,2-xylose, which both are considered immunogenic to the human immune system. In 2012 the first plant-based therapeutic was approved by the FDA. Elelyso (ProTalix BioTherapeutics), which is made in carrot cells and carry α1,3-fucose and β1,2-xylose, is intended for patients suffering from the lysosomal storage disease known as Gaucher disease. These individuals lack the enzyme glucocerebrosidase and previous treatment for this condition has been through administration of recombinant glucocerebrosidase produced from mammalian cells. As this production in mammalian cells is relatively expensive, efforts were put into producing glucocerebrosidase in a cheaper system. Remarkably, the plant-produced glucocerebrosidase does not seem to cause adverse immune reactions in humans.

Mammalian Cells as an Expression System

More than 50% of therapeutic proteins available on the market are produced using mammalian cells. Generally, mammalian expression systems are preferred for manufacture of biopharmaceuticals that are large and complex proteins and which require PTMs (most notably glycosylation) as these usually are relatively similar to proteins produced in humans. Moreover, in the case of mammalian cell lines, and animal cell lines in general, most proteins can be secreted directly into the growth medium, which is advantageous compared with bacteria/prokaryotes, where cell lysis is needed to extract protein and potential subsequent refolding of the protein. The most common mammalian (non-human) cell lines used for therapeutic protein production include murine myeloma cells (NS0 and Sp2/0), chinese hamster ovary (CHO) cells, and baby hamster kidney (BHK21) cells. However, these non-human mammalian cell lines also have disadvantages. They produce glycosylation that is not expressed in humans, more specifically galactose-α1,3-galactose (α-gal) and N-glycolylneuraminic acid (Neu5Gc). Antibodies against both of these N-glycans are found in human circulation, therefore therapeutic drugs are screened during cell line development and production for an acceptable glycan profile. Glycan profiles are considered a critical quality parameter for therapeutic proteins.

Insect Cells as an Expression System

Insect cells are easily cultured and can secrete correctly folded and posttranslationally modified proteins into the medium. The N-glycans in insect cells are, like plant N-glycans, comparable to human structures, but they are generally simpler. Most proteins produced in insect cell lines carry M3 or F(6)M3 and to some degree also high-mannose structures. The baculovirus expression system (BEVS) is the most common insect expression system and is used for many recombinant expression purposes. This insect cell based expression platform has successfully been used to produce vaccine antigens and virus-like particles. Until now, Cervarix® (GlaxoSmithKline) and FluBlok® (Protein Sciences) have been approved as vaccines by the FDA. Regulatory authorities have also approved Provenge® (Dendreon) which contains an Sf21 cell line produced protein as a component of the autologous prostate-cancer therapy product. The Spodoptera frugiperda 9 (Sf9) insect cell line has been glyco-engineered to produce more complex N-glycosylation. There are, however, still relatively high levels of F(6)M3 left as well as intermediate glycan structures.

Some insect cells, such as the Trichoplusia ni derived High Five™ and Tni PRO™ cells, glycosylate in a similar M3 structure as Sf9 and S2 cells, but with an immunogenic α1,3-linked fucose rather than a α1,6-linked as the Sf9 and S2 cells. Efforts have been made to remove fucosylation on proteins expressed in Sf9, High Five™ and Tni PRO™. The approach was not to directly target the genes responsible for the attachment of core α1,3- and α1,6-fucose, fut11/12 and FucT6 respectively, but instead targeting both α1,3- and α1,6-linked fucose at the same time by inserting a gene for an enzyme that consumes the immediate precursor to GDP-L-fucose to produce GDP-D-rhamnose, which would remove any substrate for fucose addition. This was successful, however, Mabashi-Asazuma et al saw issues with long-term stability of the cell lines.

The S2 insect cell line was originally established in 1971 by Imogene Schneider. Since then around 100 Drosophila cell lines have been obtained out of which 12 are easily cultivated. However, the primary cell lines being used for recombinant protein production are two of the original Schneider cell lines: Schneider's 2 (S2) and 3 (S3). Both S2 and S3 can be genetically modified to express recombinant proteins independent of viral infection, unlike BEVS. However, only S2 cells have been used to produce vaccine antigens for clinical trials. Stably transfected S2 cells can grow at high cell densities (up to 50×10⁶ cells/mL) in suspension and S2 based production processes are scalable. It is well established that S2 cell recombinant proteins carry pauci-mannosidic glycans and often also attach core α1,6-fucose. In addition, the present inventors have also detected small amounts of high-mannose structures and A1.

The two most prevalent N-linked glycan structures found on protein secreted from S2 and Sf9 cells are M3 and F(6)M3. In the High Five™ cell line further two structures are also found, the immunogenic F(3)M3 and F(3)F(6)M3.

To summarize, the different expression systems discussed above can be summarized as follows:

| | Expression System | | | | |
|---|---|---|---|---|---|
| | | | Plant cell | Insect cell | Mammalian cell |
| | Bacteria | Yeast | culture | culture | culture |
| Desired characteristics | | | | | |
| Cell growth | Rapid | Rapid | Slow | Slow | Slow |
| Complexity of growth | Minimum | Minimum | Complex | Complex | Complex |
| medium | | | | | |
| Cost of growth medium | Low | Low | Low | High | High |
| Expression level | High | Low to high | Low to high | Low to high | Low to moderate |
| Extracellular expression | Secretion to | Secretion to | Secretion to | Secretion to | Secretion to |
| | periplasm | medium | medium | medium | medium |
| Post tanslational | | | | | |
| modifications | | | | | |
| Protein folding | Refolding | Re folding | Proper | Proper | Proper |
| | usually | may be | folding | folding | folding |
| | required | required | | | |
| N-linked glycosylation | No | Hyper- | Simple, no | Simple, no | Complex |
| | | mannose | sialic acid, | sialic acid | |
| | | | but xylose | | |
| | | | and 1,3-fucose | | |
| O-linked glycosylation | No | Yes | Yes | Yes | Yes |
| Phosphorylation | No | Yes | Yes | Yes | Yes |
| Acetylation | No | Yes | Yes | Yes | Yes |
| Acylation | No | Yes | Yes | Yes | Yes |
| γ-Carboxylation | No | No | No | No | Yes |

Glycosylation and the Immune System

Generally, glycosylation of proteins plays an important role in various parts of vertebrate immune systems:

Antibodies, or immunoglobulins (Ig), are glycoproteins, which are produced by the immune system to target foreign invading pathogens. Igs consist of a variable antigen-binding (Fab) fragment and a constant (Fc) fragment.

Variable Fab regions bind to high diverse molecular structures in proteins, carbohydrate and lipids. Antibodies can exist in a secreted form or as membrane-bound. There are five antibody isotypes. IgA is found in mucosal areas, such as the gut, respiratory tract and urogenital tract, saliva, tears and breast milk. IgD is an antigen receptor on B-cells that have not yet been exposed to antigens. IgE acts as a receptor on the surface of mast cells and basophils and triggers histamine release from these upon cross-binding to antigens; biologically, this action protects against parasitic worms but the reaction is also involved in type I allergy. IgG consists of four different isotypes and is the major antibody involved in immunity against invading pathogens. IgM is expressed on the surface of B cells as a monomer, but also in a secreted form as a di- or pentamer, which eliminates pathogens in the early stages of the B-cell mediated humoral response before sufficient levels of IgG are reached. Core fucose on the glycan structure limits the IgG binding to the IgG Fc receptor, which results in decreased antibody-dependent cell-mediated cytotoxicity.

Antibodies are produced by the adaptive immune system, more specifically by B cells. B cells mature in the bone marrow and on release, each expresses a unique antigen-binding receptor on its membrane. When a naïve B-cell first encounters the antigen that matches its membrane-bound antibody, the binding of the antigen to the antibody (in a process the normally requires concurrent stimulation from T helper lymphocytes that recognize processed antigen presented by the B-cell on its surface) causes the B-cell to divide rapidly into memory B-cells and effector B-cells. The memory B-cells have longer life span than their parent B-cell, and they continue to express membrane-bound antibody similar to their parent B-cell. Effector cells produce the antibody in a secreted form. Effector cells only survive for a few days; however, they secrete considerable amounts of antibodies. Secreted antibodies are the major effector of humoral immunity. Some antibodies play their role simply through the binding to the target epitopes to block or induce signal transduction, whereas other antibodies bind the antigen and then recruit circulating lymphoid and myeloid cells to kill the invading pathogen by antibody-mediated effector functions (i.e., complement-dependent cytotoxicity, antibody-dependent cell-mediated cytotoxicity, and antibody-dependent cellular phagocytosis).

Dendritic cells (DCs) are the link between the innate and the adaptive immune system in mammals. Their primary function is to present digested antigens to T cells. DCs are found in tissues that are in contact with the environment, such as the skin, inner linings of nose, lungs, stomach and intestines. Once a DC is activated, it will migrate to the lymph node and interact with B and T cells. This process shapes the adaptive immune response.

The immature DCs are constantly analyzing their surrounding environment for pathogens via their pattern recognition receptors (PRRs), such as Toll-like receptors (TLRs). These recognize specific repetitive structures found on pathogens. Immature dendritic cells phagocytize pathogens and degrade these into peptides and present them on their cell surface during maturation. The surface presentation is carried out by major histocompatibility complex (MHC) molecules, which present the peptides to T-cells. During maturation, the DCs up-regulate surface receptors, such as CD80, CD86, and CD40 that greatly contribute to T-cell activation. In turn, activated T-cells aid in the full maturation of B-cells and antibody production. DCs carry certain C-type lectin receptors (CLRs) on their surface, which help instruct the DCs as when to induce immune tolerance rather than an immune reaction74. Examples of these C-type lectins are the mannose receptor (MR, CD206) and Dendritic Cell-Specific Intercellular adhesion molecule-3-Grabbing Non-integrin (DC-SIGN, CD209). Blood contains monocytes, which can be matured into DCs in vitro.

Innate immune responses are often initiated by macrophage lectins recognizing microbial glycans, which results in phagocytosis. Circulating lectins, such as serum mannose-binding lectin (MBL) and ficolins, bind to pathogen cell surfaces, hereby activating the complement cascade. When immune cells bind to glycans it can also activate intracellular signalling that either triggers or suppresses cellular responses. For example, binding of trehalose dimycolate, a glycolipid found in the cell wall of Mycobacterium tuberculosis by the macrophage C-type lectin Mincle, induces a signalling pathway that causes the macrophage to secrete pro-inflammatory cytokines. However, glycans can also have the opposite effect. For example, the B-lymphocyte carries a lectin called CD22, that when bound to α2,6-linked sialic acids initiates signalling that inhibits activation to prevent self-reactivity. Interestingly, the α2,6-linked sialic acid is also the gateway for the human influenza virus to enter human cells. The lectin of the virus, also called the hemagglutinin, facilitates binding to the host cell membrane and entry inside the cell. This interaction is highly specific. The human influenza virus recognizes α2,6-linked sialic acid and the bird influenza virus recognizes only α2,3-linked sialic acid.

The mannose receptor (MR), or Cluster of Differentiation 206 (CD206) is a C-type lectin, which is found on the surface of macrophages and dendritic cells. The MR has 8 recognition domains, which recognize terminal mannose, GlcNAc and fucose residues on glycans carried by proteins. MR has higher affinity towards branched mannose structures than linear ones, and preferably pauci-mannose structures. The MR plays a role in antigen uptake and presentation by immature DCs in the adaptive immune system. Upon binding, MR ensures delivery of the bound antigen to the early endosomes, and afterwards to the lysosomes. Here the antigen is degraded and presented on MHC class II molecules, which stimulates and polarizes the adaptive immune response.

Dendritic Cell-Specific Intercellular adhesion molecule-3-Grabbing Non-integrin, DC-SIGN, is, like MR, also a C-type lectin, which is found on the surface of macrophages and dendritic cells. This receptor also recognizes mannose, albeit DC-SIGN has higher specificity towards high-mannose, preferably M9, structures than pauci-mannose structures. DC-SIGN has been shown to bind relatively weak to F(6)M3 and to not bind F(3)M3 at all. DC-SIGN only encompasses one recognition site, however, it forms tetramers with other DC-SIGNs on the DC surface. Once DC-SIGN binds to a glycan or a microorganism it delivers the bound components to late endosomes or lysosomes, where they are destined for degradation. The degraded antigens are presented on MHC class II for T cell presentation. In specific cases it appears that both MR and DC-SIGN deliver antigens to MHC class I molecules.

Mannose Binding Lectin (MBL) is a secreted C-type lectin found in circulation, which recognizes mannose structures. Like the membrane bound MR and DC-SIGN, the recognition is not entirely specific, and MBL binds with higher specificity to high-mannose also recognizes fucose and GlcNAc. MBL encompasses a single receptor, and forms a trimer as a basic unit. When six trimmers aggregate a very strong binding is seen. In contrast to MR and DC-SIGN, MBL is capable of activating the innate immune system. Upon binding to a microorganism or antigen carrying mannose, the MBL activates the complement by the lectin pathway, followed by opsonization and phagocytosis.

Insect cells can be a good choice for expressing recombinant proteins that require correct folding and post-translational modifications, as they can express complex proteins. Insect cells naturally glycosylate in a pauci-mannosidic manner, often with a α1,6-fucose attached to the core N-acetylglucosamine (GlcNAc) and to some degree also high-mannoses. The glycosylation pathway in insect cells differ from mammalian cells, as insect cells have the fused lobes gene (fdl), which encodes a β-N-acetyl-D-hexosaminidase that cleaves off terminal GlcNAc on the A1 structure. This leads to an M3, or pauci-mannose, structure. Mammalian cells, on the other hand, retain the GlcNAc and add further carbohydrates to achieve complex glycan structures.

N-glycosylation is an important consideration in production of pharmaceuticals. Most proteinaceous drugs intended for human use show better efficacy, half-life and pharmacokinetics when carrying a humanized and complex N-glycan structure.

The importance of glycans in therapeutics is shown by the case of the cancer treatment Cetuximab, a murine myeloma cell line expressed monoclonal antibody. Cetuximab has been shown to induce allergic responses and anaphylactic shock due to the non-human glycan structure α1,3-galactose (α-gal), as 1% of circulating antibodies in humans are directed towards it. This emphasizes the importance of carefully choosing an expression system when it comes to therapeutic drugs.

To conclude, there is a continued need to provide recombinant proteins having engineered glycosylation designed for particular purposes, in particular for the purposes of avoiding undesirable immunologic reactions directed against the recombinant protein when these are administered in the form of a drug or a diagnostic means. Furthermore, the is a need to provide optimized expression systems that can supplement existing expression systems and provide for recombinant proteins with lowered or no immunogenicity in the animal receiving a dosage of the protein.

### OBJECT OF THE INVENTION

It is an object of embodiments of the invention to provide genetically modified insect cells, in particular Drosophila cells that have been engineered to produce proteinaceous expression products with a human-like N-glycosylation pattern. It is a further object of some embodiments to provide vectors and other reagents useful for this purpose. It is also an object of embodiments of the invention to provide methods for recombinant production of such expression products.

### SUMMARY OF THE INVENTION

It has been found by the present inventor(s) that is possible to genetically modify S2 cells to exhibit a human-like N-glycosylation pattern, thereby rendering the S2 system more suitable for production of protein to be used as pharmaceuticals, which are not scavenged by the immune system due to a “too foreign” glycosylation pattern.

The basic premise is that there are receptors and circulating antibodies (Abs)/lectins that target glycans and that by designing insect cell-produced antigens to exhibit a human-like glycosylation machinery that it will be possible to reduce or even abolish immunogenicity against the antigens in the recipient.

So, in a first aspect the present invention relates to an insect cell, wherein expression of the fdl gene has been disrupted.

In a sub aspect, the invention relates to a preferred version of the cell of the first aspect further comprises insertion of a functional GlcNAcT I gene and/or a functional GlcNAct II gene.

In a second aspect, the present invention relates to an insect cell, wherein expression of the fucT6 gene has been disrupted.

In a sub-aspect, the invention relates to a cell clone or cell line comprising a cell of the first and/or second aspect of the invention.

In a 3^{rd} aspect, the invention relates to an expression system comprising

1) an insect cell of the first and/or second aspects of the invention or a cell clone or cell line of the third aspect of the invention, and

2) an expression vector comprising the genetic elements necessary for effecting expression of a gene in said insect cell, where said gene has been inserted into said expression vector.

Finally, in a 4^{th} aspect, the invention relates to a method for producing an N-glycosylated polypeptide of interest, the method comprising culturing an insect cell of the first and/or second aspect of the invention or a cell clone or cell line of the fourth aspect of the invention, wherein said insect cell or a cell clone or cell line expresses a gene encoding said polypeptide, and subsequently isolating said polypeptide from the culture.

### LEGENDS TO THE FIGURE

FIG. 1: Glycans discussed in the present application.

Dark square: N-acetylglucosamine (GlcNAc), dark circle: glucose, light grey circle: mannose, triangle: fucose, light grey circle: galactose, diamond: sialic acid.

FIG. 2: Schematic presentation of different glycosylation patterns in mammalian cells.

FIG. 3: The three categories of N-Glycans, oligomannose, complex and hybrid.

Dark square: N-acetylglucosamine (GlcNAc), light grey circle: mannose, triangle: fucose, light grey circle: galactose, diamond: sialic acid.

FIG. 4: Examples of structures and their nomenclature of N-linked glycans.

Dark square: N-acetylglucosamine (GlcNAc), light grey circle: mannose, triangle: fucose, light grey circle: galactose, diamond: sialic acid.

FIG. 5: Schematic representation of the synthesis of dolichol-P-P-GlcNAc2Man9Glc3 (mature DLO).

FIG. 6: Figure of M9Glc3 degradation pathway and differentiation of insect vs. mammalian glycan shaping pathway.

FIG. 7: Chromatogram of LC-MS analysis of purified ID1-ID2a from S2-WT.

Square: GlcNAc, circle: mannose, triangle: fucose.

FIG. 8. Bar-graph representation of LC-MS analysis of purified ID1-ID2a in three different cell lines; WT, Δfdl, and Δfdl+ GlcNAcT I and GlcNAcT II.

All cell lines are monoclonal. The percentages of equal glycans, where only the core fucose differed were pooled. The label “other” refers to other glycans structures that were present in the glycan pool, but that we deemed irrelevant for the analysis of fdl disruption and insertion of GlcNAcT I and GlcNAcT II—for example higher mannose structure and a few more complex structures.

FIG. 9: Bar-graph representation of LC-MS analysis of secretome glycans of S2-WT, polyclonal ΔFucT6 (P:ΔFucT6), and monoclonal ΔFucT6 (M:ΔFucT6).

FIG. 10: Anti-human B4GT1 Western blot demonstrating production of bovine and human β-1,4-galactosyltransferase 1 in S2 cells.

FIG. 11: Lectin blot using RCA I (galactose detection) in transient transfections.—Description in Table 2.

FIG. 12: Glycoprofile of secretome of transiently transfected galactosyl transferase, galactose transporter with Glycosylation Adjust supplement added. Showing significant Galactose containing glycans (yellow circles).

FIG. 13: RCA-I lectin blot on stable cell lines of B4GT1 transfection without and with Glycosylation Adjust supplementation. All four different transfection combinations, gave higher signal when supplement is added (lanes 3-6 versus lanes 7-10) it also seems that the cell line from lane 3 has quite high galactose signal even without the supplementation. Dark bands indicates proteins with terminal galactose glycans, and each lane should be compared to the negative control in lane 1.

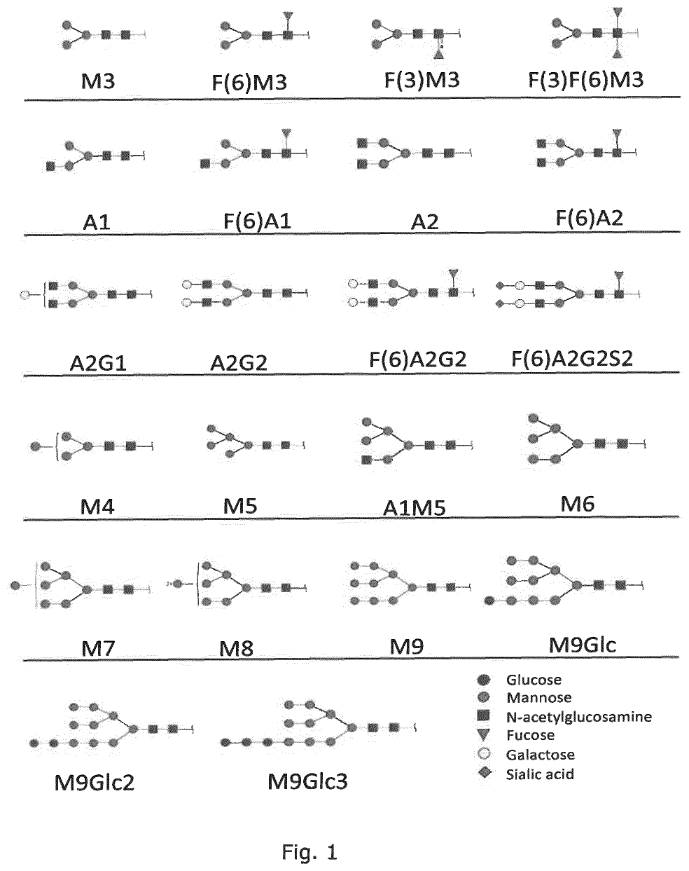

### DETAILED DISCLOSURE OF THE INVENTION

### Definitions

The term “polypeptide” is in the present context intended to mean both short peptides of from 2 to 10 amino acid residues, oligopeptides of from 11 to 100 amino acid residues, and polypeptides of more than 100 amino acid residues. Furthermore, the term is typically also intended to include proteins, i.e. functional biomolecules comprising at least one polypeptide; when comprising at least two polypeptides, these may form complexes, be covalently linked, or may be non-covalently linked. The polypeptide (s) in a protein can be glycosylated and/or lipidated and/or comprise prosthetic groups. In the present application, polypeptides and proteins are all glycosylated.

The term “subsequence” means any consecutive stretch of at least 3 amino acids or, when relevant, of at least 3 nucleotides, derived directly from a naturally occurring amino acid sequence or nucleic acid sequence, respectively.

The term “amino acid sequence” is the order in which amino acid residues, connected by peptide bonds, lie in the chain in peptides and proteins in the direction from the free N-terminus to the free C-terminus.

“Sequence identity” is in the context of the present invention determined by comparing 2 optimally aligned sequences of equal length (e.g. DNA, RNA or amino acid) according to the following formula: (N_{ref}−N_{dif})·100/N_{ref}, wherein N_{ref} is the number of residues in one of the 2 sequences and N_{dif} is the number of residues which are non-identical in the two sequences when they are aligned over their entire lengths and in the same direction. So, two sequences 5′-ATTCGGAAC-3′ and 5′-ATACGGGAC-3′ will provide the sequence identity 77.8% (N_{ref}=9 and N_{dif}=2). It will be understood that such a sequence identity determination requires that the two aligned sequences are aligned so that there are no overhangs between the two sequences: each amino acid in each sequence will have to be matched with a counterpart in the other sequence.

A “linker” is an amino acid sequence, which is introduced between two other amino acid sequences in order to separate them spatially. A linker may be “rigid”, meaning that it does substantially not allow the two amino acid sequences that it connects to move freely relative to each other. Likewise, a “flexible” linker allows the two sequences connected via the linker to move substantially freely relative to each other. In fusion proteins, which are part of the present invention, both types of linkers are useful.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule presenting the peptide.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

A “hapten” is a small molecule, which can neither induce nor elicit an immune response, but if conjugated to an immunogenic carrier, antibodies or TCRs that recognize the hapten can be induced upon confrontation of the immune system with the hapten carrier conjugate.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method disclosed herein substantially all will mount an immune response against the immunogen of the present invention.

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

“Specific binding” denotes binding between two substances which goes beyond binding of either substance to randomly chosen substances and also goes beyond simple association between substances that tend to aggregate because they share the same overall hydrophobicity or hydrophilicity. As such, specific binding usually involves a combination of electrostatic and other interactions between two conformationally complementary areas on the two substances, meaning that the substances can “recognize” each other in a complex mixture.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage—both these infectious agents are capable of introducing a heterologous nucleic acid sequence into cells.

The term “expression vector” refers to a vector containing a nucleic acid sequence coding for at least part of a gene product capable of being transcribed. In some cases, when the transcription product is an mRNA molecule, this is in turn translated into a protein, polypeptide, or peptide.

The term “expression system” denotes the combination of one or more cells/cell lines and one or more expression vectors, where the cells/cell lines can be transformed with the expression vectors and brought to produce an expression product encoded by the expression vectors.

A “glycan” is a carbohydrate or chain of carbohydrate which is linked to biomolecules (such as to lipids or proteins).

Glycobiology

Glycobiology is described as the biology, biosynthesis, structure and evolution of saccharides that are widely distributed in nature and of the proteins that recognize them. Saccharides are also called carbohydrates or sugar chains. All cells and numerous macromolecules in nature carry an array of covalently attached sugars or glycosidically linked sugar chains, which are referred to as “glycans”.

Common Monosaccharide Units of Glycoconjugates

Monosaccharides have been found in hundreds of different versions in nature. However, in common glycans the monosaccharide variation is limited to the saccharides mentioned in the following table:

| Saccharides | Explanation | Example |
|---|---|---|
| Pentoses | Five-carbon neutral sugars | D-xylose |
| Hexoses | Six-carbon neutral sugars | D-glucose |
| Hexosamines | Hexoses with an amino group | N-acetyl-D- |
| | at the 2-position, which can | glucosamine |
| | be either free or N-acetylated | |
| 6-Deoxyhexoses | | L-fucose |
| Uranic acids | Hexoses with a carboxylate | D-glucoronic acid |
| | group at the 6-position | |
| Nonulosonic | Family of nine-carbon acidic | Sialic acids |
| acids | sugars | |

Glycosidic Linkages

Monosaccharides are linked together via glycosidic bonds. The anomeric carbon of each saccharide is a stereocenter, which means that each glycosidic linkage can be constructed as either an α- or a β-linkage. Depending on which carbon atom in the sugar structure the binding occurs to, the name can be for example either Manα1,6 or Manα1,3, occurring on the 6^{th} or the 3^{rd} carbon atom respectively.

Glycan-Processing Enzymes

Generally, there are two groups of glycan-modifying enzymes the transferases and the glycosidases. The glycosyltransferases assemble branched and linear glycan chains and link monosaccharide moieties together. Glycosidases have the opposite effect; they degrade glycan structures, either for turnover of used glycans or as intermediates used as substrates in biosynthesis of glycans. The glycosyltransferases are generally specific in both donor and acceptor substrates. For example, the α2,3-sialyltransferase acts on β-linked galactose and the β1,4-galactosyltransferase acts on β-linked N-acetylglucosamine (GlcNAc).

Types of Glycosylation

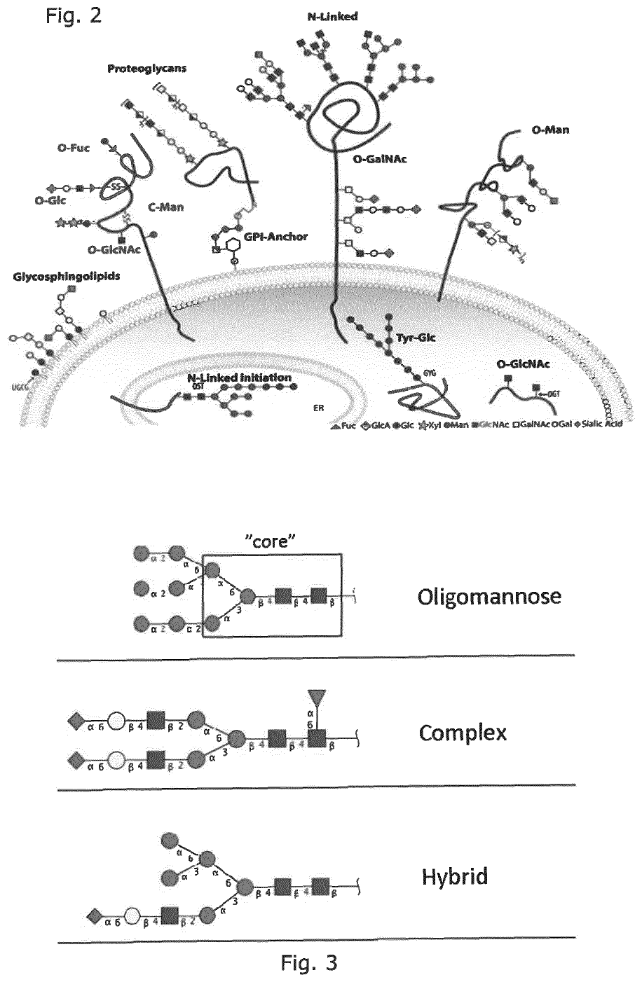

Glycosylation is a broad term and covers several different types of oligosaccharides and linkages. Glycosylation is found in all domains of life, and they vary greatly in structure across these domains. Bacteria have glycans on their surface. The most recognized is lipopolysaccharide (LPS) also known as “endotoxin” that is found on the surface of the outer membrane of Gram-negative bacteria. Gram-positive bacteria have capsular polysaccharide among other glycans on their cell wall. Archaea also carry glycans on the surface layer of their cell wall and they can even carry out N-glycosylation of proteins. The glycosylation in eukaryotic cells is more extensively studied and the major glycan-categories in mammalian cells are Glycosphingolipids, Proteoglycans, N-linked glycans, and O-linked glycans. See FIG. 2.

Glycolipids

Glycolipids are lipids with a glycan attached by a glycosidic bond. They are generally found on the extracellular surface of eukaryotic cell membranes. Here, they extend from the phospholipid bilayer and out into the extracellular space. Glycolipids maintain stability of the membrane and aid in cell-to-cell interactions. Furthermore, glycolipids can act as receptor for viruses and other pathogens to enter cells. Glycerolipids and sphingolipids are the two most common types of glycolipids.

Proteoglycans

Proteoglycans are heavily glycosylated proteins found on the extracellular side of animal cell membranes. Proteoglycans consists of a core protein and one or more covalently bound linear glycosaminoglycan chains. They fill out the space between cells in a multicellular organism and play significant roles in matrix assembly, modulation of cellular signals, and serve as a reservoir of biologically active small proteins such as growth factors.

O-Linked Glycosylation

The broad description of O-linked glycosylation is the attachment of a saccharide to an oxygen atom of an amino acid residue in a protein, most often serine and threonine. O-linked glycans are constructed by the addition of O—N-acetylgalactosamine12, O-fucose, O-glucose, O—N-acetylglucosamine or O-mannose. Hyper-O-glycosylation can result in the formation of mucin-type molecules that coat mucosal surfaces.

N-Linked Glycosylation

It is known that the structure, number, and location of N-glycans can affect the biologic activity, protein stability, clearance rate and immunogenicity of biotherapeutic proteins. N-linked glycans are most often found on cell surfaces and on secreted proteins. The N-glycosylation on proteins can occur on the amino acid sequence of a protein where an Asn precedes any amino acid but Pro, which is in turn followed by Ser or Thr. The common N-glycan “core” structure shared between all eukaryotic cells is Manα1-3(Manα1-6)Manβ1-4GlcNAcβ1-4GlcNAcβ1-Asn-X-Ser/Thr (cf. FIG. 3). Different organisms build differently onto this core structure and the glycans are categorized into 1) “oligomannoses”, where only mannose residues extend the antennas; 2) “complex”, where initially GlcNAcs extend the core; 3) “hybrid”, where Man extends the Manα1-6 arm of the core and GlcNAc extends the Manα1-3 arm. (cf. FIG. 3).

N-Linked Glycan Nomenclature in this Application

Describing N-glycans in writing can cause some confusion. The level of necessary detail and information can vary between situations. Sometimes it is necessary to know each branching and linkage type, and sometimes it is only necessary to communicate whether a structure has e.g. four or five mannoses. There has been no consensus up until recently, and authors have either invented their own nomenclature or modified an existing one. To avoid confusion, the present application will use the “Oxford notation”, which is based on building up N-glycan structures. Therefore it can be used to denote very complex glycans. In brief, the notation is as follows:

All N-glycans have two core GlcNAcs; F at the start of the abbreviation indicates a core fucose; Mx, number(x) of mannose on core GlcNAcs; Ax, number of antenna (GlcNAc) on trimannosyl core; “A2”, biantennary with both GlcNAcs as alpha1-2 linked; Gx, number (x) of linked galactose on antenna; [3]G1 and [6]G1 indicates that the galactose is on the antenna of the alpha1-3 or alpha1-6 mannose; Sx, number (x) of sialicacids linked to galactose.

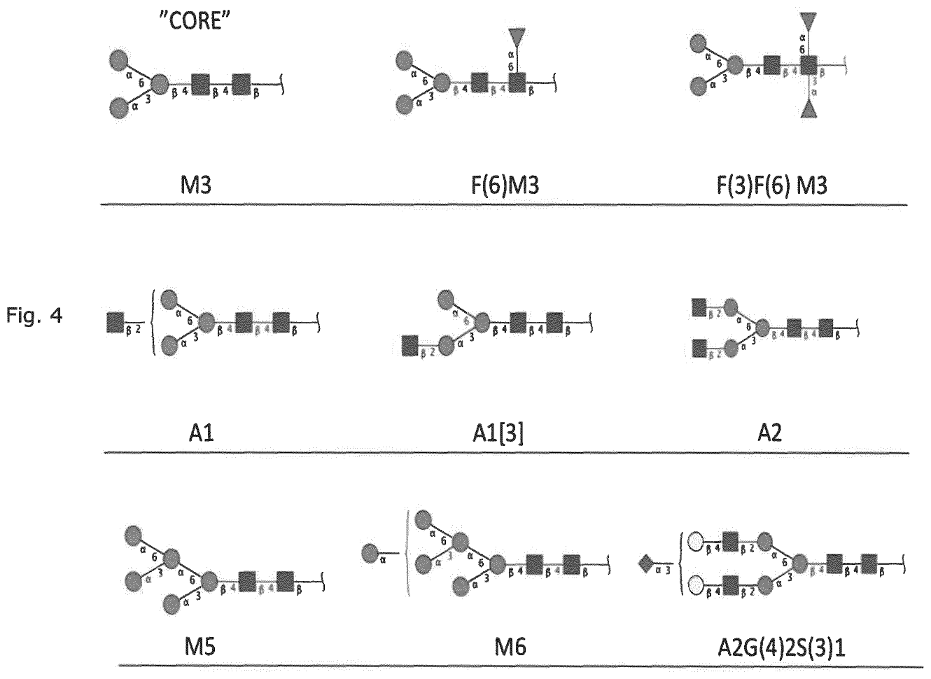

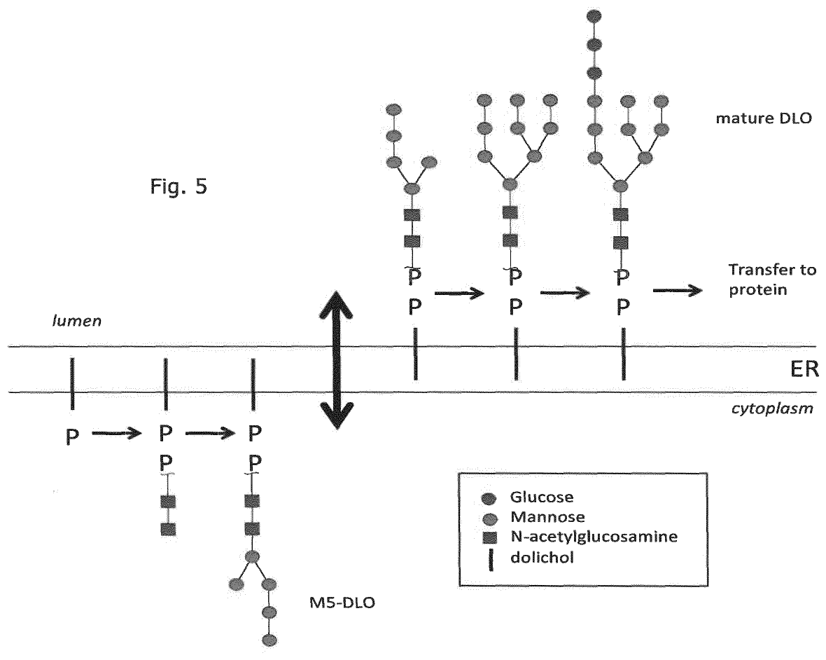

Examples of the most commonly occurring N-linked glycans in this application is given in FIG. 4.

The Oxford nomenclature is relatively intuitive. The “core” consists of two GlcNAc residues and three mannose residues. The first GlcNAc is linked to the Asn amino acid by a β-linkage. The next GlcNAc is linked by β1,4-linkage to a mannose, followed by a β1,4-linked mannose. From here, the glycan structure branches and the two remaining mannoses are attached by either an α1,3-linkage or an α1,6-linkage. This core is ubiquitous in N-glycans and is named “M3”. If it has a core fucose it is called “FM3”. If the position is known, then it is written in parenthesis e.g. “F(6)M3” in the case where the fucose is α1,6-linked, “F(3)M3” in the case where the core fucose is α1,3-linked, or e.g. “F(3)F(6) M3” in the case where the core has both a α1,3-linked and a α1,6-linked fucose. Once the sugars are added to this core, the name depends on these. The core with one GlcNAc is called “A1”. If the position is known, then it is added in square brackets, e.g. “A1[3]” if the GlcNAc is on the α1,3-linked mannose branch. If the glycans fall into the “high-mannose” category, then some structures are “fixed” both structurally and nomenclature-wise, like “M5” and for others like “M6” the position of the added mannose residue can vary. There are names for complex tri- and tetra-antennary structures, where every linkage and position is defined. An example of a more complex structure is the “A2G(4)2S(3)1”, where the “A2” describes the two β1,2-linked GlcNAcs, the “G(4)2” describes the 2 galactoses that are both β1,4-linked (and not e.g. α1,3-linked), and the “S(3)1” describes one sialic acid linked by a α2,3-link. If the position was known, then it would be indicated with a “[3]” or “[6]” referring to the α1,3- or α1,6-linked mannose branch.

N-Glycan Synthesis

The category of N-glycan that is found on a protein depends on the organism, from which it originates. All N-glycans, whether in yeast, insect cells or mammalian cells, start out as the same structure in the endoplasmic reticulum lumen. N-glycan synthesis occurs in two steps. 1) Synthesis and transfer of a dolichol-linked precursor and 2) processing steps of the Glc3Man9GlcNAc2Asn glycan.

Synthesis and Transfer of the Dolichol-Linked Precursor

The first part of N-glycosylation of a protein is the construction of the Dolichol-precursor and the attachment of this to an asparagine residue of the protein. This is described in detail below.

Dolichol phosphate is located on the cytoplasmic side of the membrane of the endoplasmic reticulum (ER). Dolichol phosphate receives GlcNAc-1-P from UDP-GlcNAc to make Dol-P-P-GlcNAc, which is then extended to Dol-P-P-M5. At this point an enzyme called “flippase” flips the structure to the inside of the ER lumen and four Man residues from Dol-P-Man and three Glc residues from Dol-P-Glc are added. This oligosaccharide is transferred to the Asn residue of a protein within the sequon N-X-S/T by an oligosaccharyltransferase that covalently binds the glycan to the protein. See FIG. 5.

Processing Steps of the M9Glc3 Glycan

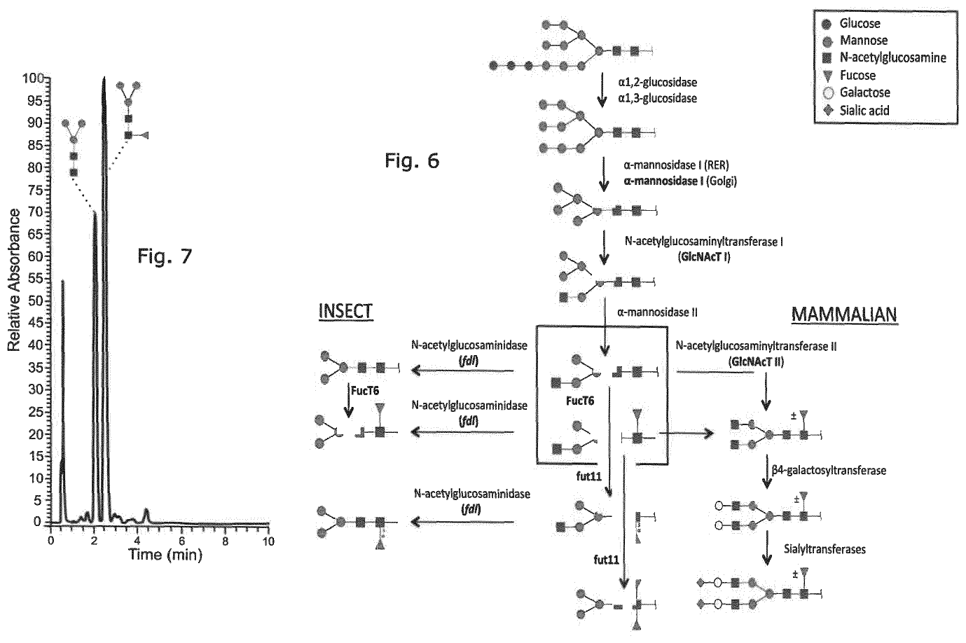

Once the protein is equipped with the M9Glc3 glycan at the N-glycan sites, the processing starts. Common for most eukaryotic organisms is that the protein is folded and transported to the Golgi apparatus for further N-linked glycan processing. Depending on the organism there are different pathways and enzymes responsible for the final N-linked glycan structure. The initial de-glucosylation is carried out by α-glucosidase I, which removes the first α1,2-linked glucose residue. The next glucose is α1,3-linked and removed by α-glucosidase II. After removal of these two glucose residues the N-linked glycan processing pathway intersects with the protein quality control pathway to ensure proper folding of the newly synthesized proteins carrying M9Glc1. The quality control pathway is mainly mediated by the ER chaperones calnexin and calreticulin. These two chaperones require the presence of the α1,3-linked glucose residue to bind to the protein. As soon the last glucose residue is removed, the chaperones terminate their folding process. This step leaves either a correctly or incorrectly folded protein with M9. The incorrectly folded proteins are re-glucosylated by a glucosyltransferase resulting in a monoglucosylated form that can again bind to chaperones. If proper folding ultimately fails the protein is degraded in a separate ER compartment, the ER-associated degradation pathway (ERAD). Correctly folded glycoproteins are finally processed by a class I α-mannosidase which removes the α1,2-mannose on the B-branch. Now, the glycoprotein, which carries M8, is transported to the Golgi where the remaining glyco-processing takes place. The proteins are delivered to the cis-side of the Golgi and are modified as they move through the medial to the trans Golgi cisternae. The pathway from now on depends on whether the N-linked glycosylation is taking place in yeast, plants, insects or mammals (FIG. 6).

This is where N-linked glycans are split up in “oligomannose”, “complex”, or “hybrid” as described in FIG. 3. Biosynthesis of complex and hybrid N-linked glycans is initiated in the medial-Golgi N-acetylglucosaminyltransferase I (GlcNAcT I), which adds GlcNAc to the second carbon atom of the α1,3-Man in the core or M5. Next, the two mannoses on the 6-branch are cleaved off by α-mannosidase II to yield A1. α-mannosidase II can only act after the action of GlcNAcT I, as it is substrate specific for A1M5. The resulting A1 is the point where invertebrates and plants start separating from mammals. The genome of plants and invertebrates, including insects, encodes a hexoaminidase by the fused lobes gene (fdl) that removes the terminal GlcNAc residue and forms M3. In contrast, the mammalian cells encode N-acetylglucosaminyltransferase II (GlcNAcT II) that adds a GlcNAc on the 6-branch and thus forms A2. This structure is then further extended to contain galactose and sialic acids. See FIG. 6. For some mammalian glycoproteins tri- or tetra-antennary structures are also found. The major core modification in both mammalian, invertebrate and plant glycans is the attachment of core fucose. In plants and some insect cells, core fucose is often added by a α1,3-linkage and in other insect cells and mammalian cells it is added by a α1,6-linkage. Similar to α-mannosidase II, α1,6-fucosyltransferase also requires the preceding action of GlcNAcT I to function. In plants, the addition of β1,2-xylose to the β-Man of the core is also common.

The following abbreviations are used throughout the present application:
α-gal: galactose-α1,3-galactose
BEVS: Baculovirus Expression Vector System
BHK21: Baby Hamster Kidney Cell
Cas9: CRISPR Associated protein 9
CE: Capillary Electrophoresis
CHO: Chinese Hamster Ovary Cells
CLR: C-type Lectin Receptor
ConA: Concanavalin A
CRISPR: Clustered Regularly Interspaced Short Palindromic Repeats
DC: Dendritic Cell
DC-SIGN: Dendritic Cell Specific Intercellular adhesion molecule-3-Grabbing Non-integrin
Ebola GP1: Ebola Glycoprotein 1
ESI: Electron spray ionization
FAB: Fast atom bombardment
Fab: Antigen binding fragment (of antibodies)
Fc: Constant fragment (of antibodies)
fdl: fused lobes gene
FucT6: α1,6-fucosylatransferase gene
fut11: α1,3-fucosylatransferase gene
GalNAc: N-acetylgalactosamine
GlcNAc: N-acetylglucosamine
GlcNAcT I: N-acetylglucosaminyl transferase I gene
GlcNAcT II: N-acetylglucosaminyl transferase II gene
HA: hemagglutinin
hEPO: human erythropoietin
HER2: Human epidermal growth factor receptor 2
HILIC: Hydrophilic interaction chromatography
HM: High-mannose
ID1-ID2a: Interdomain 1-Interdomain 2a (of VAR2CSA).
Indel: Insert/deletion
LC-MS: Liquid Chromatography Mass Spectrometry
LCA: Lens culinaris agglutinin
LPS: Lipopolysaccharide
M3 (or “Man₃”): refers to the “core” structure of glycans
mAb: monoclonal antibody
MALDI-TOF: Matrix-assisted laser desorption/ionization Time of Flight
MGAT4: N-acetylglucosaminyl transferase IV gene
MGAT5: N-acetylglucosaminyl transferase V gene
MHC: Major histocompatibility complex
mo-DC: monocyte derived dendritic cell
MPLA: Monophosphoryl lipid A
MR: Mannose receptor
MS: Mass spectrometry
Neu5Gc: N-glycolylneuraminic acid
NHEJ: Non-homologous end joining
PAM: Protospacer Adjacent Motifs
PM: Placental Malaria
PRR: Pattern recognition receptors
PTM: Post-translational modification
QIT: Quadrupole Ion Trap
S2: Drosophila melanogaster Schneider 2 cells
S3: Drosophila melanogaster Schneider 3 cells
SDS-PAGE: sodium dodecyl sulfate polyacrylamide gel electrophoresis
SPR: Surface Plasmon Resonance
TLR: Toll-like Receptor
VAR2CSA: Receptor of malaria infected erythrocytes, which bind to placenta cells
VLP: Virus-like particle
WT: Wild-type

### SPECIFIC EMBODIMENTS OF THE INVENTION

1^{st} Aspect and 2^{nd} Aspect—the Genetically Modified Insect Cells of the Invention

Genetically modified insect cells disclosed herein are useful as organisms for producing polypeptides. The first aspect relates to an insect cell, wherein expression of the fdl gene has been disrupted, which has, as shown herein in the examples, the consequence that the FM3/M3 glycan structures are reduced in proportion on expressed protein. For the first aspect of the invention, it is hence preferred that the N-glycosylated protein produced by the cell of the first aspect exhibit less than 40% FM3/M3 glycan structures. Another consequence is that the cells attains FA1/A1 glycan structures, which are not found in the unmodified parent cells. Hence according to the present invention, it is preferred that the cell of the first aspect produces N-glycosylated protein comprising at least 2% FA1/A1 glycan structures, but it is—as shown herein—possible to obtain much higher amounts of FA1/A1 in protein produced by the cell: the cell of the first aspect preferably produces N-glycosylated protein comprising at least 5% FA1/A1 glycan structures, such as at least 10%, at least 15%, at least 20%, at least 25%, at least 30%, at least 35%, at least 40%, at least 45%, at least 50%, and at least 55%. Percentages of about 55, 56, 57, 58, and 59 are preferred, if the no further modifications beyond disruption of fdl have been introduced.

A preferred version of the cell of the first aspect further comprises insertion of a functional GlcNAcT I gene and/or a functional GlcNAct II gene. This has the effect of further modifying the phenotype of the cell towards one exhibited by human cells, when it comes to N-glycosylation. Preferred cells of the first aspect are those that produce N-glycosylated protein comprising less than 15% (such as <14%, <13%, <12%, <11%, and <10%) FM3/M3 glycan structures (i.e. a further reduction than what the fdl disruption alone provides), less than 30% (such as <29%, <28%, <27%, <26%, <25%, <24%, and <23%) FA1/A1 glycan structures, and more than 50% (such as >51%, >51%, >53%, >54%, >55%, >56%, >57%, >58%, >59%, >60%, >61%, >62%, >63%, and >64%) FA2/A2 glycan structures. Particularly preferred cells produce N-glycosylated protein comprising 8-12% FM3/M3, 20-24% FA1/A1, and 62-66% FA2/A2 glycan structures, such as about 9.7 FM3/M3, about 22.5% FA1/A1, and about 64.5% FA2/A2.

In this context, the “fdl gene” is a term which also is meant to cover beta-hexosaminidase encoding genes in other insect cells than Drosophila melanogaster derived cells. In fact, the term “fdl gene” in the present context covers not only the gene in D. melanogaster, which encodes beta-hexosaminidase, but any equivalent gene encoding beta-hexosaminidase in other insect cells.

In the second aspect, the invention relates to a genetically modified insect cell where the expression of the FucT6 gene has been disrupted, which has the effect of blocking attachment of core α1,6-fucose to protein produced by the cells. In fact, it is preferred that the N-glycosylated protein produced by the cells of the second aspect comprises less than 2%, e.g. preferably 0%, core α1,6-fucose.

In this context, the “FucT6 gene” is a term which also is meant to cover alpha-(1,6)-fucosyltransferase encoding genes in other insect cells than Drosophila melanogaster derived cells. In fact, the term “FucT6 gene” in the present context covers not only the gene in D. melanogaster, which encodes alpha-(1,6)-fucosyltransferase, but any equivalent gene encoding alpha-(1,6)-fucosyltransferase in other insect cells.

In particular preferred embodiments of the invention, cells having the characteristics of both the first and second aspect are contemplated, i.e. insect cells with disruption of both the fdl gene and the FucT6 gene.

The insect cells of the both the first and second aspect (and their combination) can be selected from any insect cell useful for recombinant protein production. Preferred insect cells are selected from the group consisting of Drosophila Schneider's cells such as S2 and S3, Spodoptera frugiperda cells, such as Sf9 and derivates thereof (e.g. Mimic™ cells and ExpiSf9 cells) and Sf21, and Trichoplusia ni cells such as BTI-Tn-5B1-4 (High Five™ cells), and Tn-368. However, insect cells of particular interest are Drosophila cells, such as S2, S3 cell, and S2R+ cells.

As indicated in the examples, in order to further “humanize” the proteins produced by the insect cells of the 1^{st} and 2^{nd} aspects of the invention, they may further comprise introduction of a number of functional genes that express enzymes of importance for N-glycosylation. For instance, the cells may comprise a functional gene expressing a β-1,4-galactosyltransferase, and/or a functional gene expressing an α2,3-sialyltransferase and/or a functional gene expressing an α2,6-sialyltransferase and/or a functional gene expressing at least one sialic acid transporter protein and/or a functional Mgat4 gene and/or a functional Mgat5 gene. As indicated, each of these further genetic modifications can appear alone or in combination with any one of or more of the other modifications.

In addition to these modifications the cells of the first and second aspects can further include a heterologous polynucleotide which expresses a heterologous protein (such as an antibody or other therapeutically relevant compound), where the expression product of the heterologous protein can be N-glycosylated in a desired way due to the cell's modifications. In this context, cf. the description of expression systems below.

In some embodiments the cells of the first and second aspects are provided as a cell clone or cell line, comprising the cell.

For production purposes, it is advantageous that the genetically modified cell disclosed herein is stably transformed by having those nucleic acids that are introduced and which are disclosed above stably integrated into its genome, and in certain embodiments it is also preferred that the genetically modified cell secretes or carries on its surface the glycosylated heterologous polypeptide disclosed herein, since this facilitates recovery of the heterologous polypeptides produced.

As noted above, stably genetically modified cells are preferred—these i.a. allows that cell lines comprised of genetically modified cells as defined herein may be established—such cell lines are particularly preferred aspects of the invention.

Further details on cells and cell lines are presented in the following:

Techniques for recombinant gene production, introduction into a cell, and recombinant gene expression are well known in the art. Examples of such techniques are provided in references such as Ausubel, Current Protocols in Molecular Biology, John Wiley, 1987-2002, and Greene and Sambrook: “Molecular Cloning: A Laboratory Manual (Fourth Edition)”, Cold Spring Harbor Laboratory Press (ISBN-10: 9781936113422).

As used herein, the terms “cell”, “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A genetically modified cell includes the primary subject cell and its progeny.

Host cells are in the present application of insect cell origin. Several cell lines and cultures are available for use as a host cell, and they can be obtained through the American Type Culture Collection (ATCC), which is an organization that serves as an archive for living cultures and genetic materials or from other depository institutions such as Deutsche Sammlung vor Micrroorganismen und Zellkulturen (DSM). An appropriate host can be determined by one of skill in the art based on the vector backbone and the desired result. A plasmid or cosmid, for example, can be introduced into a prokaryote host cell for replication of many vectors or expression of encoded proteins.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

3^{rd} Aspect—Expression Systems

Numerous expression systems exist that comprise at least a part or all of the compositions discussed above. Eukaryote-based systems can be employed for use with the present invention to produce N-glycosylated polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ Baculovirus expression system from CLONTECH®.

According to the invention, an expression system of the 3^{rd} aspect comprises

1) a genetically modified insect cell disclosed above or a cell clone or cell line comprising the modified insect cell, and

2) an expression vector comprising the genetic elements necessary for effecting expression of a (heterologous) gene in said insect cell, where said gene has been inserted into said expression vector. In other words, the cells of the present invention can function together with appropriately selected expression vectors comprising a gene of interest, and any of a number of commercially available expression vectors can hence be used to transfect/transform the cells of the present invention to allow subsequent production and purification of the expression product from the gene of interest.

4^{th} Aspect—Protein/Polypeptide Production

In this method for producing an N-glycosylated polypeptide of interest, which comprises culturing a genetically modified insect cell of the invention or a cell clone or cell line comprising the insect cell, wherein said insect cell or a cell clone or cell line expresses a gene encoding said polypeptide, and subsequently isolating said polypeptide from the culture, any conventional culturing system and purification method can be used. For the provision of the recombinantly transfected cell, also any useful method for transfection of insect cells is applicable. Thus, in general the present invention provides for optimized insect cells that are useful in recombinant production of N-glycosylated protein which has a humanized or human-like N-glycosylation pattern.

Compositions Enabled by the Invention

Pharmaceutical compositions disclosed herein may either be prophylactic (i.e. suited to prevent disease) or therapeutic (i.e. to treat disease).

Such compositions typically comprise immunising N-glycosylated polypeptide(s), protein(s) or peptide(s), usually in combination with “pharmaceutically acceptable carriers”, which include any carrier that does not itself induce the production of antibodies harmful to the individual receiving the composition or targeting the protein/pathogen. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles.

Such carriers are well known to those of ordinary skill in the art. Additionally, these carriers may function as immunostimulating agents (“adjuvants”) in cases where this is relevant.

Pharmaceutical compositions typically will contain diluents, such as water, saline, glycerol, ethanol, etc. Additionally, auxiliary substances, such as wetting or emulsifying agents, pH buffering substances, and the like, may be present in such vehicles.

The compositions—since they contain pharmaceutically active polypeptides—are prepared as injectables, either as liquid solutions or suspensions; solid forms suitable for solution in, or suspension in, liquid vehicles prior to injection may also be prepared. The preparation also may be emulsified or encapsulated in liposomes for enhanced effect.

Compositions comprise an effective and pharmaceutically acceptable amount of polypeptides, as well as any other of the above-mentioned components, as needed. By “effective amount”, it is meant that the administration of that amount to an individual, either in a single dose or as part of a series, is effective for treatment or prevention as the case may be. This amount varies depending upon the health and physical condition of the individual to be treated, the taxonomic group of individuals to be treated (e.g. nonhuman primate, primate, etc.), the formulation of the active principle, the treating doctor's assessment of the medical situation, and other relevant factors. It is expected that the amount will fall in a relatively broad range that can be determined through routine trials. For administration of therapeutic (humanized) antibodies, the dosing range for humans is typically rather flexible—a review over rational dosing of therapeutic MAbs can be found in Bai S et al. (2012), Clin Pharmacokinet. 51(2): 199-135.

The compositions are conventionally administered parenterally, eg, by injection, either subcutaneously, intramuscularly, or transdermally/transcutaneously, intraveneously, intraarterially, intrathecally. Additional formulations suitable for other modes of administration include oral, pulmonary and nasal formulations, suppositories, and transdermal applications.

Dosage treatment may be a single dose schedule or a multiple dose schedule. The composition may be administered in conjunction with other agents such as immunoregulatory agents.

Pharmaceutical compositions comprise polypeptides/proteins whose production is disclosed herein. The pharmaceutical compositions will comprise a therapeutically effective amount thereof.

The term “therapeutically effective amount” or “prophylactically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. Reference is however made to the ranges for dosages of immunologically effective amounts of polypeptides, cf. above.

However, the effective amount for a given situation can be determined by routine experimentation and is within the judgement of the clinician.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N. J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

### Example 1

Preparation of “Humanized” S2 Cell Lines

Plasmid Construction

The online E-CRISP tool (www.e-crisp.org; German Cancer Research Center) was used to identify CRISPR/Cas9 sequences within the Drosophila melanogaster genome that target fdl (UniProt: Q8WSF3) and FucT6 (UniProt: Q9VYV5). sgRNA target sequences were selected as 20 nt sequences preceding an NGG PAM sequence in the genome. The oligonucleotide pairs XX-F and XX-R were used to construct DNA fragments consisting of each targeting sequence with overhangs to enable their subcloning into pExpreS²-CRISPR (ExpreS²ion Biotechnologies, Hørsholm, Denmark).

The sequences of the synthetic oligos are as follows:

| Name | Sequence of oligo |
|---|---|
| Fdl3-F | TTCGCGGCGCAGCGATACAGCCA |
| | (SEQ ID NO: 1) |
| Fdl3-R | AACTGGCTGTATCGCTGCGCCGC |
| | (SEQ ID NO: 2) |
| Fdl12-F | TTCGCTGGGCCTTGGTGACTCCT |
| | (SEQ ID NO: 3) |
| Fdl12-R | AACAGGAGTCACCAAGGCCCAGC |
| | (SEQ ID NO: 4) |
| FucT6_ | TTCGTATCGCCGATCGAGTTGGCC |
| 36-F | (SEQ ID NO: 5) |
| FucT6_ | AACGGCCAACTCGATCGGCGATAC |
| 36-R | (SEQ ID NO: 6) |
| FucT6_ | TTCGAGTTAATTGAGACTATGCAC |
| 71-F | (SEQ ID NO: 7) |
| FucT6_ | AACGTGCATAGTCTCAATTAACTC |
| 71-R | (SEQ ID NO: 8) |
| FucT6_ | TTCGCAAGGAACGGGGCTCCGAAC |
| 56-F | (SEQ ID NO: 9) |
| FucT6_ | AACGTTCGGAGCCCCGTTCCTTGC |
| 56-R | (SEQ ID NO: 10) |

N-acetylglucosaminyl transferases I and II were constructed by ordering Drosophila codon optimized GlcNAcT I (NP_525117, NCBI) and GlcNAcT II (GenBank: CAC83074.1) (GeneArt, ThermoFisher) and inserting them into the pExpreS2-PAC plasmid (ExpreS²ion Biotechnologies, Hørsholm, Denmark) using restriction enzymes EcoRI and NotI.

S2 Cell Culture, Transfection, and Cloning

ExpreS² Cells (ExpreS²ion Biotechnologies, Hørsholm, Denmark), hereafter “S2 cells”, were routinely maintained at 25° C. and 130 rpm in suspension in 125 ml shake flasks (vented cap) in culture medium EX-CELL 420 Serum-Free Medium for Insect Cells (Sigma-Aldrich, cat. Nr. 14420C, Steinheim, Germany) supplemented with 100 units/mL penicillin and 0.1 mg/mL streptomycin (Pen-Strep Solution, Biological Industries, cat. 03-031B, Cromwell, Conn., USA), hereafter “culture medium”. The S2 cells were counted with a CASY® Cell Counter every 3-4 days and passaged by centrifugation or dilution to 8×10⁶ cells/ml.

For transfection, the S2 cells were passaged to 8×10⁶ cells/ml in shake flasks in culture medium and transfected the following day, by splitting the cells to 2×10⁶ cells/ml and mixing with first 50 μl ExpreS² Insect-TRx5 (ExpreS²ion Biotechnologies, Hørsholm, Denmark) transfection reagent and second with 12.5 μg of plasmid DNA. The transfected cells were then transferred to a 25 cm² tissue culture flask (In Vitro, Fredensborg, Denmark). A polyclonal cell line was selected herein for 21 days in culture medium supplemented with 10% fetal bovine serum (FBS) (Fischer Scientific, Roskilde, Denmark) and 1.5 mg/ml zeocin (Thermo Fisher, Hvidovre, Denmark) or 4.0 mg/ml geneticin (InvivoGen, Toulouse, France), by dilution to 1×10⁶ cells/ml every 3-4 days or whenever the cells reached a density higher than 1.5×10⁶ cells/ml.

For selection with puromycin (GlcNAcT I and GlcNAcT II), the cells were transfected and grown in a 50 ml vented Falcon Tube in 8 ml of culture medium. The cells were transfected by adding and swirling 100 μl ExpreS² Insect-TRx5 Transfection Reagent and 20 μg of plasmid DNA. After 2-4 hours FBS was added to a concentration of 10%. Puromycin was added after 24 hours to a final concentration of 100 μg/ml. The cells were counted and split by centrifugation every 3-4 days in culture medium+10% FBS+100 μg/ml puromycin for 2 weeks. Hereafter the stable cell lines were transferred to a 125 ml shake flask and passaged as described previously.

Monoclonal cell lines were obtained by limited dilution in 96 well plates (In Vitro, cat. GR-655180, Fredensborg, Denmark) using non-transfected S2 feeder cells at 0.6×10⁶ cells/ml. Stably transfected polyclonal cells were seeded out at concentrations of 100 cells/mL, 30 cells/mL or 10 cells/mL. The total volume in wells was 150 μl culture medium supplemented with 10% fetal bovine serum and 1.5 mg/ml zeocin or 4.0 mg/ml geneticin. Over 2-3 weeks the 96-well plates were inspected regularly, and monoclonal cell lines were identified and expanded from 96 well plates to 250 mL shake flasks (Sigma-Aldrich, cat. CLS431255, St. Louis, Mo., USA).

For production of ID1-ID2a (Nielsen M A et al. (2015), PLoS ONE 10:1-12, doi:10.1371/journal.pone.0135406), the cells were expanded to a total volume of 2 L and grown in a 5 L Thomson Optimum Growth Flask (Thomson, Sittingbourne, England) and 200 μl/L PD30 was added. The cell culture supernatant was harvested by centrifugation at 4400 xG, filtered through a 0.22 μm filter, concentrated 1:4 times by tangential flow filtration, and exchanged into 20 mM Phosphate, pH 6.6, using a tangential flow filtration device (Pall, N.Y., USA). Protein purification of ID1-ID2a proceeded through ion exchange chromatography (HiTrap SP Sepharose FF, GE Healthcare Life Sciences) and was eluted step-wise (6%, 15%, and 22%) in 20 mM Phosphate and 1 M NaCl pH 6.6. The purified protein was diluted 1:4 in 20 mM Phosphate pH 7.0 and run on a HiTrap Capto adhere column (GE Healthcare Life Sciences, Brøndbyvester, Denmark) for removal of contaminants, and was eluted step-wise (13%, 30%, 80%) in 20 mM Phosphate, 1 M NaCl pH 7.0 and was aliquoted and then snap-frozen in liquid nitrogen and stored at −80° C.

Indel Detection by Amplicon Analysis (IDAA)

Genomic DNA was extracted using PureLink™ Genomic DNA Mini Kit (Fisher Scientific, cat. nr. K182001, Roskilde, Denmark). Primers were designed using the online prediction tool “Primer3”: FucT6 (F:TTCGCAAGGAACGGGGCTCCGAAC (SEQ ID NO:9), R:GCAAGGAACGGGGCTCCGAACGTT, (SEQ ID NO:11)). PCR was performed using Phusion High-Fidelity PCR kit (1×HF buffer, 0.2 mM dNTP, 0.25 U Phusion polymerase, 0.025 μM forward primer, 0.25 μM reverse primer and 0.25 μM 6-FAM 5′-labelled universal primer (Thermofisher, cat. nr. F553S, Hvidovre, Denmark)). The PCR-amplicons were analyzed by fragment length analysis (Eurofins, Glostrup, Denmark).

SDS-PAGE and Lectin Blotting

Supernatant samples were analyzed by SDS-PAGE and Western Blot analysis. Briefly, proteins were resolved by 10% SDS-PAGE and then transferred to a nitrocellulose membrane. Non-specific binding was blocked by incubating the membrane in Carbo-Free™ Blocking Solution (VectorLabs, Cat. No. SP-5040, Burlingame, Calif., USA) for 30 minutes at room temperature. Then the blot was incubated for 30 minutes in PBS with 10 μg/ml biotinylated lectin and washed in PBS+0.2% Tween 20™. The secondary antibody was HRP-conjugated Streptavidin (Fisher Scientific, Roskilde, Denmark) diluted 1:5000 for 45 min followed by a wash in PBS+0.2% Tween 20™. Novex® ECL (WP20005, Fisher Scientific, Roskilde, Denmark) was used for detection. Lectin used: for recognition of α1,6-fucose: Biotinylated Lens culinaris (LCA) from VectorLabs, Burlingame, Calif., USA.

Glycoprofiling

Glycoprofiling was performed as previously described in Grav L M et al. (2015), Biotechnology Journal 10:1446-56, doi:10.1002/biot.201500027. Briefly, supernatants were filtered and proteins contained in the sample were concentrated by centrifugation using Amicon Ultra columns (Merck Millipore, Merck KGaA, Darmstadt, Germany) with 3000 Da cutoff. N-glycans from retained proteins were released and fluorescently labeled with GlycoPrep Rapid N-Glycan kit (ProZyme Inc., Hayward, Calif.) or GlycoWorks RapiFluor-MS N-Glycan Kit (Waters, Elstree, UK). Labelled N-glycans were analyzed by LC-MS on a Thermo Ultimate 3000 HPLC with fluorescence detector coupled on-line to a Thermo Velos Pro Ion Trap MS. Glycan abundance was measured by integrating the areas under normalized fluorescence spectrum peaks with Xcalibur software (Thermo Fisher Scientific, Hvidovre, Denmark) giving the relative amount of the glycans. All annotated sugar structures are peaks with correct mass and at least a signal to noise value of 10:1 as calculated with Xcalibur.

Results

Initial Glycan Profiling and Glycan Consistency Test Showed Two Main N-Glycans

The work on a humanized glycan structure was carried out in a monoclonal S2 cell line that recombinantly expresses the placental malaria protein VAR2CSA truncation variant ID1-ID2a. VAR2CSA has been shown to bind to many cancer cell types and can reduce or clear the cancer cells efficiently in mouse models, when coupled to a toxin. One way to optimize this protein for future cancer therapy could be to reduce the immunogenicity by adding human-like glycans to it. Therefore, it was attempted to humanize the glycan structure on ID1-ID2a. The glycosylation of wild-type S2 cell expressed ID1-ID2a was analyzed on Liquid Chromatography-Mass Spectrometry (LC-MS) prior to any modifications and showed two main peaks of 36% M3 and 58% FM3 (FIG. 7), as is commonly seen for insect cells.

Next it was confirmed that the glycosylation profile did not change significantly under different growth conditions and purification protocols. Purified ID1-ID2a from production batches grown for three or four days, from shake flasks and bioreactors, and during different steps of the purification, was analyzed. The results (data not shown) were consistent: M3 with or without core fucose in an approximately 40:60 ratio, very similar to the profile seen in FIG. 7.

Humanization: Disruption of fdl and Insertion of GlcNAcT I and GlcNAcT II

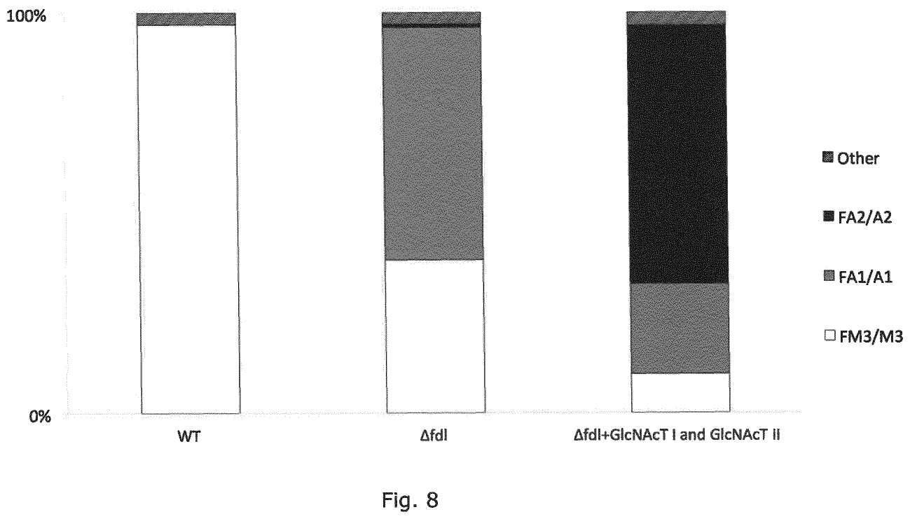

In an effort to achieve a more humanized glycan structure, disruption of fdl was aimed at. For this, two different target sgRNAs were chosen (cf. above under “plasmid construction”) and stable cell lines were established for both targets in parallel. One monoclonal cell line (henceforth called Δfdl) showed an increase of FA1/A1 from 0% to 58% on LC-MS (FIG. 8), which indicated a phenotypical effect of the disruption.

The second step towards humanizing this cell line was to transfect it with GlcNAcT I and GlcNAcT II expression plasmids, which were responsible for attaching GlcNAcs to the mannose tree (Δfdl+ GlcNAcT I and GlcNAcT II ID1-ID2a). Purified ID1-ID2a from Δfdl+ GlcNAcT I and GlcNAcT II ID1-ID2a was analyzed on LC-MS (FIG. 8).

In the WT ID1-ID2a cell line there was >97% FM3/M3 glycans. After disruption of fdl and re-cloning, there was a significant decrease from >97% FM3/M3 to <39% and an increase of FA1/A1 to 58%. According to a previously suggested glycosylation pathway in Sf9 insect cells it should only be necessary to insert the N-acetylglucosaminyl transferases II (GlcNAcT II), since the insect cells already harbor GlcNAcT I. Shi X and Jarvis D L (2013), Current Drug Targets 8:1116-25. However, since >38% FM3/M3 was observed after the disruption of fdl, both GlcNAcT I and GlcNAcT II were incorporated into the genome of this monoclonal cell line and re-cloned. Hereafter, <10% FM3/M3, <22% FA1/A1, and >64% FA2/A2 were found on purified ID1-ID2a as depicted in FIG. 8. The “other” section of the bar graphs in this figure represents glycans that occur in <2% of the total glycans and comprises M5 (0.5%), A3 (0.5%), M6 (0.7%), M7 (0.4%), G1 (0.5%), FG1 (0.5%), and A2G(4)2Ga1S1/Deoxynonulosonate-FG2/M5A2G1S1 (0.2%).

This Δfdl+ GlcNAcT I and GlcNAcT II ID1-ID2a cell line is a solid foundation for building the next steps in constructing humanized glycosylation on ID1-ID2a.

Glycan Analysis by LC-MS of the Impact of the FucT6 Disruption

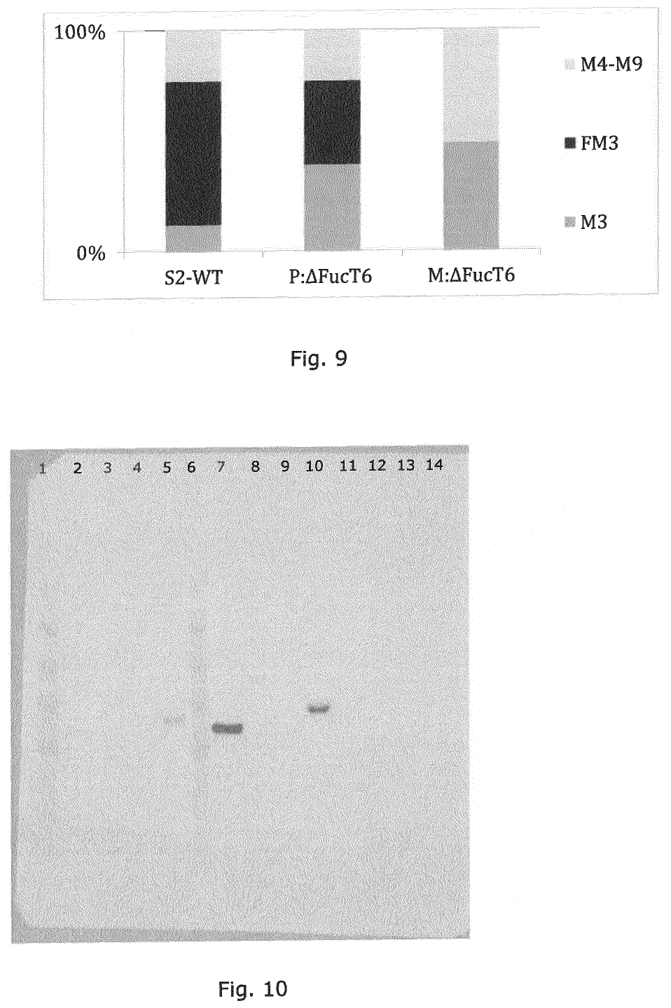

It was also of interest to construct a cell line that does not attach core α1,6-fucose to the glycan, as this allows production of antibodies with enhanced effector functions. In order to achieve this, the goal was to disrupt FucT6, which encodes an α1,6-fucosyltransferase. The work on disrupting the FucT6 gene was conducted in a wild type cell line (S2-WT). Three CRISPR/Cas9 sgRNA target sequences were designed for the disruption of the FucT6 gene (cf. above under “plasmid construction) and these were transfected in parallel in S2 cells to establish stable cell lines. All supernatant proteins, or the secretome, of S2-WT and the ΔFucT6 with a polyclonal disruption were analyzed on LC-MS (FIG. 9)

In contrast to the fdl disruption in the WT-ID1-ID2a cell line described above, it was possible to see an effect of the disruption on a polyclonal level (FIG. 9). The S2-WT cell line showed approximately >64% of FM3 and <12% of M3. After disruption of the FucT6 gene the polyclonal glycan profile shifted and showed a decrease in FM3 to <37% and an increase in M3 to >39% of the total glycans. This indicated that the disruption was successful. However, to establish a monoclonal cell line with no display of fucose a further round of sub-cloning was performed. A total of 39 ΔFucT6 clones were obtained by limited dilution. These were screened by a lectin blot and analyzed for genotypic indels by IDAA. Based on the lectin blot two clones were chosen for LC-MS analysis, and both showed similar secretome glycan patterns with 0% fucose on the glycans upon LC-MS. One example is shown in (FIG. 9). This clone shows a complete phenotypical knockout of the fucosyltransferase encoded by FucT6.

Other insect cell lines have been shown to carry core α1,3-linked fucose. To investigate if the cells had any α1,3-fucose in the glycan pool a Western Blot on the full secretome was carried out with an α1,3-fucose specific antibody, and a α1,3-fucose positive control: no such structures (data not shown) was detected. The monoclonal cell line with a disrupted FucT6 gene can now serve the basis of future antibody and Fc-fusion protein expression in S2 cells for instance in combination with the humanized glycomodifications discussed above.

### DISCUSSION

Two new cell lines were constructed: 1) Partial humanization by disruption of fdl and insertion of GlcNAcT I and GlcNAcT II and 2) Disruption of the FucT6 gene to produce antibodies and Fc-fusion proteins.

The first glycan-modification we performed was disruption of fdl and insertion of GlcNAcT I and GlcNAcT II to achieve a more human-like glycosylation on ID1-ID2a. Previously, fdl has been inhibited by addition of GlcNAcase inhibitor (2-acetamido-1,2-dideoxynojirimycin), by RNA interference or by attempting to outcompete it by inserting glycosyltransferases. On a monoclonal level it was observed that the CRISPR/Cas9 editing of the fdl locus reduced or limited the FDL function. Although the disruption strategy worked, approximately 38% of the native FM3/M3 glycan structures remained on ID1-ID2a expressed in Δfdl (FIG. 9). Similar levels of residual pauci-mannose have been seen before; also in a Drosophila fly where there was a complete disruption of the fdl gene—this means that the result is not merely the consequence of a limited edit of the fdl leaving where parts of the enzyme are still active.

Furthermore, after addition of GlcNAcT I and GlcNAcT II and after cloning, FA2/A2 was present >64% on purified ID1-ID2a. This appears to be the highest percentage FA2/A2 reported on any insect cell produced protein. The closest to a humanized structure reached in insect cell culture is that of Toth A M et al. (2014), Journal of Biotechnology 182-183:19-29. doi:10.1016/j.jbiotec.2014.04.011, who managed to achieve 39% terminally sialylated N-glycans on purified hEPO.

In an effort to humanize the N-glycosylation of S2 cells, Kim et al. (Kim Y K et al. (2011), Journal of Biotechnology 153:160-6, doi:10.1016/j.jbiotec.2011.02.009) RNAi suppressed fdl and inserted the β1,4-galactosyltransferase into the genome. In this cell line they reached small amounts of G2 on purified recombinant hEPO. Kim et all also found that by addition of β1,4-galactosyltransferase without the addition of GlcNAcT I and GlcNAcT II there was an increase in the level of G1 on hEPO (Kim Y K et al. (2009), Glycobiology 19:301-8, doi:10.1093/glycob/cwn138). We only found these glycan structures present on ID1-ID2a after insertion of GlcNAcT I and GlcNAcT II and cloning. This could both be due to differences in glycosylation sites in the recombinant proteins and differences in the S2 cell lines as these adapt, grow and glycosylate differently over time in different laboratories. The glycan profile for the Δfdl+ GlcNAcT I and GlcNAcT II ID1-ID2a cell line also included small amounts of a glycan with m/z: 1203.5. This mass corresponds to a FG2 with a glycolyl deoxynonulosonate on the one branch. This has not previously been reported in S2 cells or other insect cells.

It is contemplated that the next steps for modification of this cell line towards an even more human-like glycan structure will be introduction of a gene expressing a β1,4-galactosyltransferase. Furthermore, after obtaining a G2 glycan structure, it then follows to additionally introduce genes expressing α2,3-sialyltransferase and α2,6-sialyltransferase, and sialic acid transporter proteins, to achieve fully humanized glycan structures. It could also be of value to express Mgat4 and Mgat5 to make tri- and tetra-antennary structures. However, it is expected that glycans of >64% FA2/A2 will induce immunological advantages in a mouse cancer model and increase serum half-life compared to native ID1-ID2a.

The second modification performed was the disruption of the FucT6 gene. The FucT6 gene was successfully disrupted in a WT S2 cell line by the use of CRISPR/Cas9. The polyclonal pool showed an effect and the analyzed clone showed 100% absence of α1,6-fucose. Some insect cell lines, such as the High Five cell line, are known to express the immunogenic α1,3-fucose. Even though small amounts of di-fucosylated glycans have been detected and even less with only the α1,3-fucose in the Drosophila embryo, no 1,3-fucose or double-fucosylated glycans was detected in the WT-S2 cells or the ΔFucT6 clone upon western blot analysis with an α1,3-fucose specific antibody.

It does not appear that a disruption of FucT6 in S2 cells or in other insect cell lines has been published; this unique cell line creates a strong foundation for the expression of antibodies in S2 cells and for expressing Fc-fusion proteins due to the enhanced effector functions that the fucose-lacking glycan structures facilitate.

### Example 2

G2F Cell Line (Biantennary Galactose)

S2 cells transfected with ExpreS2 vector harboring gene encoding Cas9 and guide RNA targeting fdl (knockout of β-N-acetylhexosaminidase, G418 selection), genes encoding N-acetylglucosaminyltransferase I and N-acetylglucosaminyltransferase II (Drosophila Mgat1 and Mgat2, puromycin selection). This polyclonal cell line has been cloned by serial dilution resulting in cell line with ca 65% biantennary GlcNAcs (G0, data already submitted for initial filing).

This monoclonal cell line was transfected with bovine β-1,4-galactosyltransferase 1 from Bos indicus (B4GT1, accession number XP_019821962) and the one from human (B4GalT1, NP_001488) where CTS (cytoplasmic/transmembrane/stem) domain was exchanged for the one of human FUT7 gene CTS (Genbank accession number NP_004470, amino acids 1-48) as done by Geisler et al. (2014) in ‘Engineering β-1,4-galactosyltransferase I to reduce secretion and enhance N-glycan elongation in insect cells’. This has shown shift of the enzyme being present in pellet instead of supernatant (anti human galactosyl transferase Western blot, FIG. 10.).

TABLE 1
list of samples in blot in FIG. 10.

| | | Expression | | | kDa |
|---|---|---|---|---|---|
| 1 | Ladder | | | | |
| 2 | B4GT1_bovin, pExpreS2-blast | Transient | Sup. | Cleaved (CAT | 31.4 |
| | | | | only) | |
| 3 | | Transient | Pellet | Uncleaved | 44.8 |
| 4 | FUT7(CTS)-B4GT1_bovin(CAT), pExpreS2- | Transient | Sup. | Cleaved (CAT | 31.4 |
| | Blast | | | only) | |
| 5 | | Transient | Pellet | Uncleaved | 37.0 |
| 6 | Ladder | | | | |
| 7 | B4GalT1_human, pExpreS2-Blast | Transient | Sup. | Cleaved (CAT | 31.4 |
| | | | | only) | |
| 8 | | Transient | Pellet | Uncleaved | 43.9 |
| 9 | FUT7(CTS)-B4GalT1_human(CAT), | Transient | Sup. | Cleaved (CAT | 31.4 |
| | pExpreS2-Blast | | | only) | |
| 10 | | Transient | Pellet | Uncleaved | 36.9 |
| 11 | | | | | |
| 12 | | | | | |
| 13 | WT (no GalT) | — | Sup. | Control | |
| 14 | | — | Pellet | Control | |

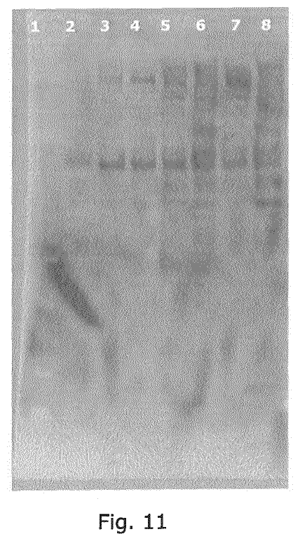

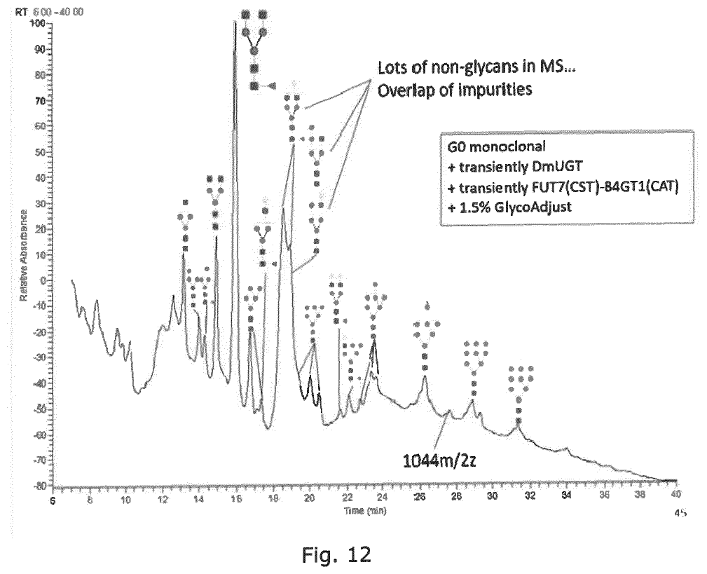

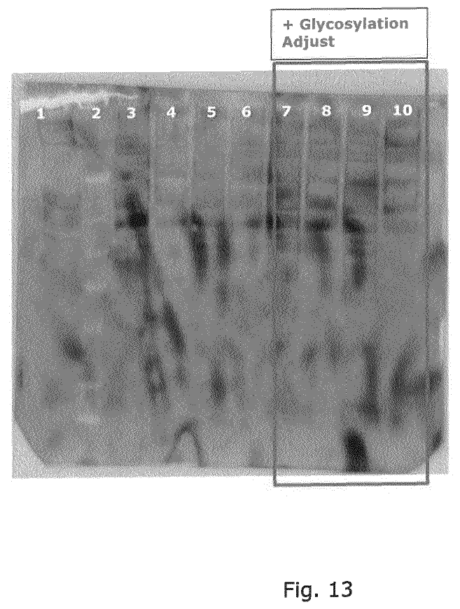

For detection of galactose we used Ricinus Communis lectin (RCA I, Vector Biolabs) that is specific to galactose. We also used EX-CELL® Glycosylation Adjust (Gal+) protein quality supplement to increase occupancy of galactoses (the producer does not specify what is inside the supplement). In FIG. 11. we can see that the signal for galactose (dark bands indicating proteins with terminal galactose glycans) is significantly stronger for transient transfections with supplement addition (lane 5 versus lane 6 and lane 7 versus lane 8). The glycoprofile of transiently transfected galactosyl transferase, galactose transporter with Glycosylation Adjust supplement added can be seen in FIG. 12. RCA I lectin blot on samples from stable clones can be seen in FIG. 13. In the latter, there is a stronger signal after addition of Glycosylation Adjust.

TABLE 2
List of samples in lectin blot in FIG. 11.

| | | Expression | Additive |
|---|---|---|---|
| 1 | Ladder | — | — |
| 2 | S2 WT | — | — |
| 3 | G0 negative control | Stable, | |
| | (passaged as for | monoclonal | |
| | transient transf.) | | |
| 4 | GFP transfection control | Transient | — |
| 5 | B4GT1_bovin | Transient | |
| 6 | B4GT1_bovin | Transient | +Glycosylation Adjust |
| 7 | FUT7(CTS)- | Transient | |
| | B4GalT1_human(CAT) | | |
| 8 | FUT7(CTS)- | Transient | +Glycosylation Adjust |
| | B4GalT1_human(CAT) | | |

TABLE 3
List of samples from lectin blot from FIG. 13.

| | | Expression | | Additive |
|---|---|---|---|---|
| 1 | FUT7(CTS)-B4GT1_bovine(CAT), | Transient | in G0 monoclone | — |
| | pExpreS2-Blast | | | |
| 2 | Ladder | | | — |
| 3 | FUT7(CTS)-B4GT1_bovine(CAT), | Stable | In Δfdl clone 1 | — |
| | pExpreS2-1 | | | |
| | Mgat1 + Mgat2, pExpreS2-PAC | | | |
| 4 | FUT7(CTS)-B4GT1_bovine(CAT), | Stable | In Δfdl clone 6 | — |
| | pExpreS2-1 | | | |
| | Mgat1 + Mgat2, pExpreS2-PAC | | | |
| 5 | FUT7(CTS)-B4GT1_bovine(CAT), | Stable | In Δfdl clone 1 | — |
| | pExpreS2-1 | | | |
| | Mgat1, pExpreS2-PAC | | | |
| | Mgat2, pExpreS2-PAC | | | |
| 6 | FUT7(CTS)-B4GT1_bovine(CAT), | Stable | In Δfdl clone 6 | — |
| | pExpreS2-1 | | | |
| | Mgat1, pExpreS2-PAC | | | |
| | Mgat2, pExpreS2-PAC | | | |
| 7 | FUT7(CTS)-B4GT1_bovine(CAT), | Stable | In Δfdl clone 1 | +Glycosylation Adjust |
| | pExpreS2-1 | | | |
| | Mgat1 + Mgat2, pExpreS2-PAC | | | |
| 8 | FUT7(CTS)-B4GT1_bovine(CAT), | Stable | In Δfdl clone 6 | +Glycosylation Adjust |
| | pExpreS2-1 | | | |
| | Mgat1 + Mgat2, pExpreS2-PAC | | | |
| 9 | FUT7(CTS)-B4GT1_bovine(CAT), | Stable | In Δfdl clone 1 | +Glycosylation Adjust |
| | pExpreS2-1 | | | |
| | Mgat1, pExpreS2-PAC | | | |
| | Mgat2, pExpreS2-PAC | | | |
| 10 | FUT7(CTS)-B4GT1_bovine(CAT), | Stable | In Δfdl clone 6 | +Glycosylation Adjust |
| | pExpreS2-1 | | | |
| | Mgat1, pExpreS2-PAC | | | |
| | Mgat2, pExpreS2-PAC | | | |

1. An insect cell, wherein expression of the fdl gene has been disrupted.

2. The insect cell according to claim 1, which produces N-glycosylated protein with less than 40% FM3/M3 glycan structures.

3. The insect cell according to claim 1, which produces N-glycosylated protein comprising at least 2% FA1/A1 glycan structures.

4. The insect cell according to claim 1 or 2, comprising insertion of a functional GlcNAcT I gene and/or a functional GlcNAcT II gene.

5. The insect cell according to claim 4, which produces N-glycosylated protein comprising less than 15% FM3/M3 glycan structures, less than 30% FA1/A1 glycan structures, and more than 50% FA2/A2 glycan structures.

6. An insect cell, wherein expression of the FucT6 gene has been disrupted.

7. The insect cell according to claim 6, which produces N-glycosylated protein comprising less than 2%, preferably 0%, core α1,6-fucose.

8. The insect cell according to claim 6 or 7, which further is as defined in any one of claims 1-5.

9. The insect cell according to any one of the preceding claims which is selected from the group consisting of Drosophila cells, such as S2 and S3, Spodoptera frugiperda cells, such as Sf9 (and derivatives thereof) and Sf21, and Trichoplusia ni cells such as BTI-Tn-5B1-4 and Tn-368.

10. The insect cell according to claim 9, which is a Drosophila cell, such as an S2 or S3 cell.

11. The insect cell according to any one of the preceding claims, which further comprises a functional gene expressing a β-1,4-galactosyltransferase.

12. The insect cell according to any one of the preceding claims, which further comprises a functional gene expressing an α2,3-sialyltransferase.

13. The insect cell according to any one of the preceding claims, which further comprises a functional gene expressing an α2,6-sialyltransferase.

14. The insect cell according to any one of the preceding claims, which further comprises a functional gene expressing at least one sialic acid transporter protein.

15. The insect cell according to any one of the preceding claims, which further comprises a functional Mgat4 gene.

16. The insect cell according to any one of the preceding claims, which further comprises a functional Mgat5 gene.

17. A cell clone or cell line, comprising the cell according to any one of the preceding claims.

18. An expression system comprising
1) an insect cell according to any one of claims 1-16 or a cell clone or cell line according to claim 17 and
2) an expression vector comprising the genetic elements necessary for effecting expression of a gene in said insect cell, where said gene has been inserted into said expression vector.

19. A method for producing an N-glycosylated polypeptide of interest, the method comprising culturing an insect cell according to any one of claims 1-16 or a cell clone or cell line according to claim 17, wherein said insect cell or a cell clone or cell line expresses a gene encoding said polypeptide, and subsequently isolating said polypeptide from the culture.


SEQUENCE LISTING (11 sequences)

SEQ ID NO: 1 - DNA, 23 bp, Artificial Sequence
  Prime sequence
ttcgcggcgcagcgatacagcca

SEQ ID NO: 2 - DNA, 23 bp, Artificial Sequence
  Prime sequence
aactggctgtatcgctgcgccgc

SEQ ID NO: 3 - DNA, 23 bp, Artificial Sequence
  Prime sequence
ttcgctgggccttggtgactcct

SEQ ID NO: 4 - DNA, 23 bp, Artificial Sequence
  Prime sequence
aacaggagtcaccaaggcccagc

SEQ ID NO: 5 - DNA, 24 bp, Artificial Sequence
  Prime sequence
ttcgtatcgccgatcgagttggcc

SEQ ID NO: 6 - DNA, 24 bp, Artificial Sequence
  Prime sequence
aacggccaactcgatcggcgatac

SEQ ID NO: 7 - DNA, 24 bp, Artificial Sequence
  Prime sequence
ttcgagttaattgagactatgcac

SEQ ID NO: 8 - DNA, 24 bp, Artificial Sequence
  Prime sequence
aacgtgcatagtctcaattaactc

SEQ ID NO: 9 - DNA, 24 bp, Artificial Sequence
  Prime sequence
ttcgcaaggaacggggctccgaac

SEQ ID NO: 10 - DNA, 24 bp, Artificial Sequence
  Prime sequence
aacgttcggagccccgttccttgc

SEQ ID NO: 11 - DNA, 24 bp, Artificial Sequence
  Prime sequence
gcaaggaacggggctccgaacgtt